(19) United States
(12) Patent Application Publication
(10) Pub. No.: US 2003/0092759 A1
(43) Pub. Date: May 15, 2003

(54) Anticonvulsant derivatives useful for the treatment of restless limb syndrome and periodic limb movement disorder

(76) Inventor: Faruk S. Abuzzahab SR., Minneapolis, MN (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/252,814

(22) Filed: Sep. 23, 2002

Related U.S. Application Data
(60) Provisional application No. 60/324,440, filed on Sep. 24, 2001.

(51) Int. Cl. A61K 31/7008; A61K 31/35; A61K 31/255

(52) U.S. Cl. 514/459; 514/23; 514/464; 514/517

(57) ABSTRACT

Anticonvulsant derivatives useful for treating restless limb syndrome, more particularly restless legs syndrome, restless arms syndrome, periodic limb movement disorder and associated sleep disturbances, regardless of underlying cause.

### BACKGROUND OF THE INVENTION

The present invention is directed to anticonvulsant derivatives useful in the treatment of restless limb syndrome and periodic limb movement disorder, including restless legs syndrome, restless arm syndrome, periodic limb movement disorder and associated sleep disturbances.

Compounds of Formula I:

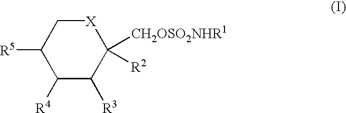

are structurally novel antiepileptic compounds that are highly effective anticonvulsants in animal tests (MARYANOFF, B. E, NORTEY, S. O., GARDOCKI, J. F., SHANK, R. P. AND DODGSON, S. P. J. Med. Chem. 1987, 30, 880-887; MARYANOFF, B. E., COSTANZO, M. J., SHANK, R. P., SCHUPSKY, J. J., ORTEGON, M. E., AND VAUGHT J. L. Bioorg. Med. Chem. Lett. 1993, 3, 2653-2656; SHANK, R. P., GARDOCKI, J. F., VAUGHT, J. L., DAVIS, C. B., SCHUPSKY, J. J., RAFFA, R. B., DODGSON, S. J., NORTEY, S. O., MARYANOFF, B. E. Epilepsia 1994, 35, 450-460; MARYANOFF B E, COSTANZO M J, NORTEY S O, GRECO M N, SHANK R P, SCHUPSKY J J, ORTEGON M P, VAUGHT J L. J. Med. Chem. 1998, 41, 1315-1343). These compounds are covered by three U.S. Pat. No. 4,513,006, No. 5,242,942, and No. 5,384,327. One of these compounds 2,3:4,5-bis-O-(1-methylethylidene)-β-D-fructopyranose sulfamate, known as topiramate, has been demonstrated in clinical trials of human epilepsy to be effective as adjunctive therapy or as monotherapy in treating simple and complex partial seizures and secondarily generalized seizures (E. FAUGHT, B. J. WILDER, R. E. RAMSEY, R. A. REIFE, L D. KRAMER, G. W. PLEDGER, R. M. KARIM et. al., Epilepsia 1995, 36 (S4), 33; S. K. SACHDEO, R. C. SACHDEO, R. A. REIFE, P. LIM and G. PLEDGER, Epilepsia 1995, 36 (S4), 33; T. A. GLAUSER, Epilepsia 1999, 40 (S5), S71-80; R. C. SACHDEO, Clin. Pharmacokinet. 1998, 34, 335-346), and is currently marketed for the treatment of seizures in patients with simple and complex partial epilepsy and seizures in patients with primary or secondary generalized seizures in the United States, Europe and most other markets throughout the world.

Compounds of Formula I were initially found to possess anticonvulsant activity in the traditional maximal electroshock seizure (MES) test in mice (SHANK, R. P., GARDOCKI, J. F., VAUGHT, J. L., DAVIS, C. B., SCHUPSKY, J. J., RAFFA, R. B., DODGSON, S. J., NORTEY, S. O., and MARYANOFF, B. E., Epilepsia 1994, 35, 450-460). Subsequent studies revealed that Compounds of Formula I were also highly effective in the MES test in rats. Topiramate was also found to effectively block seizures in several rodent models of epilepsy (J. NAKAMURA, S. TAMURA, T. KANDA, A. ISHII, K. ISHIHARA, T. SERIKAWA, J. YAMADA, and M. SASA, Eur. J. Pharmacol. 1994, 254, 83-89), and in an animal model of kindled epilepsy (A. WAUQUIER and S. ZHOU, Epilepsy Res. 1996, 24, 73-77).

Restless limb syndrome (RLS), also known as Ekborn's syndrome, is a common, chronic disorder which causes symmetric and/or asymmetric dysesthesia to the lower extremities during rest or sleep. Restless movement of the arms, may also occur. Symptom's include involuntary, rhythmic reaction movements occurring at night, during sleep stage I and sleep stage II. Sleep is disturbed and is followed by daytime fatigue. It is mostly a primary or hereditary disease, but may be associated with uremia, diabetes, rheumatoid arthritis, primary amyloidosis or malignancy. Clinical examination may reveal evidence of underlying systemic disease or mild peripheral neuropathy but is more often normal. The pathology of RLS is still unclear, however various factors include malfunction of the dopamine and opiate receptors in the central nervous system. Symptoms of restless legs syndrome may respond to correction of coexisting iron-deficiency anemia or to treatment with dopaminergic medications such as levodopa, bromocriptine, and the like; benzodiazepines such as diazepam, clonazepam, and the like; or opiates such as codeine, propoxyphene, oxycodone, and the like.

In addition, the unpredictable movements associated with restless limb syndrome will also impact spouses sufferers, resulting in disturbed sleep and the potential for injury as a result of the uncontrolled movements. Frequently, the couple will resort to sleeping in separate beds, thus significantly affecting their quality of life.

The primary or first-line treatments for RLS include sedative/hypnotic medications including benzodiazepines, such as triazolam, temazepam, flurazepam, quazepam, estazolam, alprazolam, diazepam, clonazepam, lorazepam, oxazepam, zolpidem, zaleplon and zopiclone; dopaminergic drugs such as carbidopa/levodopa, Sinemet, pergolide, bromocriptine, selegiline, pramipexole, ropinirole, cabergoline, tolcapone, entacapone and amantadine; and analgesic medications such as propoxyphene, codeine, Tylenol with codeine, pentazocine, hydrocodone, oxycodone, hydromorphone, meperidine, fentanyl, methadone, morphine, levorphanol tartrate and tramadol.

Secondary treatment options include anti-seizure medications such as gabapentin, carbamazepine, divalproex sodium and primidone; hypertensive medications such as clonidine, propranolol and diltiazem; multiple sclerosis medications such as lioresal; and antidepressant medications such as amoxapine, amitriptyline, perphenazine, chlordiazepoxide, desipramine, nortriptyline, doxepin, trimipramine, imipramine, perphenazine, protriptyline, phenazine, tranylcypromine, venlafaxine, paroxetine, fluoxetine, nefazodone, sertraline, citalopram, maprotiline, trazodone, bupropion, fluvoxamine maleate, clomipramine and mirtazepine. (The Southern California RLS Support Group, Treatment Page, www.rlshelp.org: Adler C H, Clin. Neuropharmacol., 1997, 20 (2), 148-151; Merren M D, South Med. J., 1998, 91 (8), 739-44; Wetter T C, Pollmacher T, J. Neurol., 1997, 244 (4 Suppl 1), S37-45).

The use of caffeine has been noted to intensify RLS symptoms. In fact, it is often recommended that RLS sufferers avoid methylxanthines-containing products specifically caffeinated beverages such as coffee or soft drinks and theophylline-containing beverages such as tea as well as amine-containing foods such as chocolate. The consumption of alcohol has also been associated with increases in the span or intensity of symptoms for most individuals. Non-pharmacological therapies including supplements of iron, folic acid, vitamin B12 and magnesium have also been suggested. Researchers have also recently found that RLS may be worsened or caused by an underlying lack of adequate iron stores, which can be measured by a blood sample to check ferritin levels. If ferritin levels are found to be less than 50 mcg/L, supplementation with iron may prove to be beneficial.

Various drugs have also been reported as exacerbating RLS. These drugs include calcium-channel blockers used to treat high blood pressure and heart conditions, Reglan metoclopramide, some antinausea medications, some cold and allergy medications, major tranquilizers including haloperidol and phenothiazines, and the anti-seizure medication phenytoin. Although some patients have reported improvement in their symptoms of RLS with use of antidepressive medications, it is more often the case that the use of antidepressive medications worsens the symptoms of RLS. (Restless Legs Syndrome Foundation Homepage, FAQ, www.rls.org). For example, a recent case study report an increase in RLS symptoms associated with the antidepressant sertraline. (Hargrave, R. and Beckley, D. J., Psychosomatics, 39(2), 1998, pp177-178).

Periodic limb movement disorder (PLMD) or periodic limb movements in sleep syndrome is a different disorder from RLS. PLMD exhibits as periodic limb movements defined as stereotyped, periodic movements of the legs and/or upper limbs during sleep. PLMD may or may not cause arousals or awakenings during sleep (Trenkwalder C, Walters A S, Hening W, Neurol Clin 1996,14(3):629-50; Krueger BR, Mayo Clin Proc 1990, 65(7):999-1006; Picchietti D L, Walters A S, Sleep 1996, 9(9):747-8; Kageyama T, Kabuto M, Nitta H, Kurokawa Y, Taira K, Suzuki S, Takemoto T, Psychiatry Clin Neurosci 2000, 54(3):296-8). Current treatment options for PLMD are similar to those used for the treatment of RLS (Saletu M, Anderer P, Saletu-Zyhlarz G, Prause W, Semler B, Zoghlami A, Gruber G, Hauer C, Saletu B, Eur Neuropsychopharmacol 2001, 11(2):153-61; Chesson A L Jr, Wise M, Davila D, Johnson S, Littner M, Anderson WM, Hartse K, Rafecas J, Sleep 1999, 1;22(7):961-8; Hening W, Allen R, Earley C, Kushida C, Picchietti D, Silber M, Sleep 1999, 1;22(7):970-99; Ehrenberg B L, Eisensehr I, Corbett K E, Crowley P F, Walters A S, J Clin Psychopharmacol 2000, 20(5):574-8).

It has now been found that compounds of formula I as herein defined are useful in the treatment of restless limb syndrome and periodic limb movement disorder and associated sleep disturbances, regardless of the underlying cause.

In an embodiment of the present invention is the treatment of restless limb syndrome. In another embodiment of the present invention is the treatment of drug-induced or drug-exacerbated restless limb syndrome.

In another embodiment of the present invention is the treatment of periodic limb movement disorder. In another embodiment of the present invention is the treatment of drug-induced or drug-exacerbated periodic limb movement disorder.

In another embodiment of the present invention is the treatment of sleep disturbances associated with restless limb syndrome or periodic limb movement disorder.

### DISCLOSURE OF THE INVENTION

Accordingly, it has been found that compounds of the following formula (I):

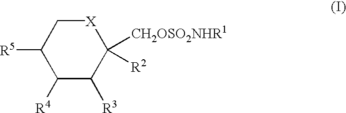

wherein X is O or CH₂, and R¹, R², R³, R⁴ and R⁵ are as defined hereinafter are useful in restless legs syndrome, restless arm syndrome and associated sleep disturbances.

### DETAILED DESCRIPTION OF THE PREFERRED EMBODIMENTS

As used herein, the term “restless limb syndrome” shall include restless legs syndrome (Ekborn's Syndrome), restless arms syndrome and associated sleep disturbances, regardless of underlying cause.

As used herein, the term “periodic limb movement disorder” shall mean the condition wherein a subject experiences and/or exhibits stereotyped, periodic movements of the legs and/or upper limbs during sleep.

As used herein, the term “drug induced or exacerbated restless limb syndrome” shall mean restless leg, restless arm and associated sleep disorders whose cause or severity was triggered or may be traced to drug treatment with selective serotonin reuptake inhibitors or other serotonergic agents.

As used herein, the term “drug induced or exacerbated periodic limb movement disorder” shall mean periodic limb movement disorders whose cause or severity was triggered or may be traced to drug treatment with selective serotonin reuptake inhibitors or other serotonergic agents.

As used herein, the term “subject” refers to an animal, preferably a mammal, most preferably a human, who has been the object of treatment, observation or experiment.

As used herein, the term “therapeutically effective amount” means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

The sulfamates of the invention are of the following formula (I):

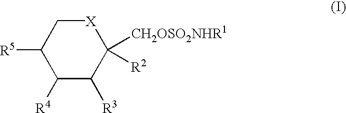

wherein

X is CH₂ or oxygen;

R¹ is hydrogen or alkyl; and

R², R³, R⁴ and R⁵ are independently hydrogen or lower alkyl and, when X is CH², R⁴ and R⁵ may be alkene groups joined to form a benzene ring and, when X is oxygen, R² and R³ and/or R⁴ and R⁵ together may be a methylenedioxy group of the following formula (II):

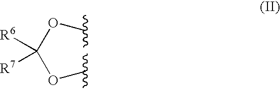

wherein

R⁶ and R⁷ are the same or different and are hydrogen, lower alkyl or are alkyl and are joined to form a cyclopentyl or cyclohexyl ring.

R¹ in particular is hydrogen or alkyl of about 1 to 4 carbons, such as methyl, ethyl and iso-propyl. Alkyl throughout this specification includes straight and branched chain alkyl. Alkyl groups for R², R³, R⁴, R⁵, R⁶ and R⁷ are of about 1 to 3 carbons and include methyl, ethyl, iso-propyl and n-propyl. When X is CH₂, R⁴ and R⁵ may combine to form a benzene ring fused to the 6-membered X-containing ring, i.e., R⁴ and R⁵ are defined by the alkatrienyl group ═C—CH═CH—CH═.

A particular group of compounds of formula (I) is that wherein X is oxygen and both R² and R³ and R⁴ and R⁵ together are methylenedioxy groups of the formula (II), wherein R⁶ and R⁷ are both hydrogen both alkyl or combine to form a spiro cyclopentyl or cyclohexyl ring, in particular where R⁶ and R⁷ are both alkyl such as methyl. A second group of compounds is that wherein X is CH₂ and R⁴ and R⁵ are joined to form a benzene ring. A third group of compounds of formula (I) is that wherein both R² and R³ are hydrogen.

The compounds of formula (I) may be synthesized by the following methods:

(a) Reaction of an alcohol of the formula RCH₂OH with a chlorosulfamate of the formula ClSO₂NH₂ or ClSO₂NHR¹ in the presence of a base such as potassium t-butoxide or sodium hydride at a temperature of about −20° to 25° C. and in a solvent such as toluene, THF, or dimethylformamide wherein R is a moiety of the following formula (III):

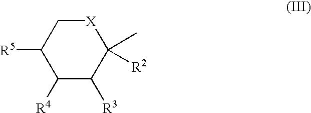

(b) Reaction of an alcohol of the formula RCH₂OH with sulfurylchloride of the formula SO₂Cl₂ in the presence of a base such as triethylamine or pyridine at a temperature of about −40° to 25° C. in a solvent such as diethyl ether or methylene chloride to produce a chlorosulfate of the formula RCH₂OSO₂Cl.

The chlorosulfate of the formula RCH₂OSO₂Cl may then be reacted with an amine of the formula R¹NH² at a temperature of abut 40° to 25° C. in a solvent such as methylene chloride or acetonitrile to produce a compound of formula (I). The reaction conditions for (b) are also described by T. Tsuchiya et al. in Tetrahedron Lett., 1978, 3365.

(c) Reaction of the chlorosulfate RCH₂OSO₂Cl with a metal azide such as sodium azide in a solvent such as methylene chloride or acetonitrile yields an azidosulfate of the formula RCH₂OSO2N₃ as described by M. Hedayatullah in Tetrahedron Lett. 1975, 2455. The azidosulfate is then reduced to a compound of formula (I) wherein R¹ is hydrogen by catalytic hydrogenation, e.g. with a noble metal and H₂ or by heating with copper metal in a solvent such as methanol.

The starting materials of the formula RCH₂OH may be obtained commercially or as known in the art. For example, starting materials of the formula RCH₂OH wherein both R² and R³ and R⁴ and R⁵ are identical and are of the formula (II) may be obtained by the method of R. F. Brady in Carbohydr. Res. 1970, 14, 35 or by reaction of the trimethylsilyl enol ether of a R⁶COR⁷ ketone or aldehyde with fructose at a temperature of about 25° C., in a solvent such a halocarbon, e.g. methylene chloride in the presence of a protic acid such as hydrochloric acid or a Lewis Acid such as zinc chloride. The trimethylsilyl enol ether reaction is described by G. L. Larson et al. in J. Org. Chem. 1973, 38, 3935.

Further, carboxylic acids and aldehydes of the formulae RCOOH and RCHO may be reduced to compounds of the formula RCH₂OH by standard reduction techniques, e.g. reaction with lithium aluminum hydride, sodium borohydride or borane-THF complex in an inert solvent such a diglyme, THF or toluene at a temperature of about 0° to 100° C., e.g. as described by H. O. House in “Modern Synthetic Reactions”, 2nd Ed., pages 45 to 144 (1972).

The compounds of formula I: may also be made by the process disclosed U.S. Pat. No. 4,513,006, No. 5,242,942, and No. 5,384,327, which are incorporated by reference herein.

The compounds of formula I include the various individual isomers as well as the racemates thereof, e.g., the various alpha and beta attachments, i.e., below and above the plane of the drawing, of R², R³, R⁴ and R⁵ on the 6membered ring. Preferably, the oxygen of the methylenedioxy group (II) are attached on the same side of the 6-membered ring.

As used herein abbreviations are as defined below:

H.S. = hour of sleep (at bedtime)
b.i.d. = bis in diem (twice daily)
Prn = per necessitatem (as needed)

The ability of the compounds of formula I to treat restless legs syndrome, restless arm syndrome and associated sleep disorders is based on the results of recent clinical case studies, as described in more detail below.

### EXAMPLE 1

In the first case, a female patient had exhibited recurrent depression and restless legs syndrome (RLS) since the early age of 16 years. Prior to treatment, within 15 minutes of falling asleep, the patient was awakened by uncontrollable jerking of her legs. For five years, the patient had been treated with fluoxetine HCl at 60 mg, but the drug only produced unwanted side effects, such as agitation and loss of sexual desire. The patient had also used lorazepam and alprazolam to treat the RLS, with some benefit.

The patient was switched to topiramate, initially at 25 mg, with a gradual increase in dosage to 75 mg/day for treatment of the RLS. Sertraline HCl at 100 mg/day was added for the treatment of her concurrent depression. Lorazepam and alprazolam were discontinued. The RLS and depression symptoms were both reduced.

### EXAMPLE 2

The patient was a 40 year old male who had a lifelong chaotic sleep/wake schedule disorder, depression and RLS. The patient was initially treated with trixenryphenidyl at 5 mg/day for the RLS, but he experienced side effects including dry mouth, blurred vision and amnesia. The patient was then switched to clonazepam at 2-4 mg/day, with very little positive effect on the RLS symptoms. In the previous 15 years, the patient had also used carisoprodol, chlordiazepoxide HCl, clorazepate dipotassium, meprobamate, phenobarbital, flurazepam HCl, promethazine, levodopa and carbidopa, with some benefit in controlling the RLS.

Topiramate was started at 25 mg/day and increased gradually to 300 mg/day during a six month period. The patient reported that the restless legs syndrome symptoms were successfully reduced.

### EXAMPLE 3

The patient was a 44 year old male, with diagnosed recurrent unipolar depression, post traumatic stress disorder, panic disorder, nicotine, alcohol and cannabis dependency. The patient also complained of migraine headaches and restless leg syndrome.

The patient was started at 25 mg HS topiramate, with dosage increased to 100 mg HS and then further increased to a final dosage of 200 mg HS. Concurrent pharmacotherapy included clonazepam at 2 mg/day, desipramine HCl at 10 mg/day, clonidine at 0.3 g b.i.d., mirtazepine at 15 mg HS, triamcinolone acetonide prn, albuterol prn, potassium at 99 m, A to Z multivitamin 1×/day and fluticasone propionate prn.

The patient reported that topiramate at 100 mg HS was as effective as clonazepam 6 mg HS for restless leg syndrome and that the two drugs in combination were more effective than either drug alone for RLS. The patient also reported that topiramate was more effective than carisoprodol for RLS.

Thus, for treating restless legs syndrome, a compound of formula (I) may be employed by administering repeated oral doses in the range of about 10 to 650 mg once or twice daily, preferably in the range of about 25 to about 325 mg daily.

Optimal dosages to be administered may be readily determined by those skilled in the art, and will vary with the particular compound used, the mode of administration, the strength of the preparation and the advancement of the disease condition. In addition, factors associated with the particular patient being treated, including patient's sex, age, weight, diet, time of administration and concomitant diseases, will result in the need to adjust dosages.

To prepare the pharmaceutical compositions of this invention, one or more sulfamate compounds of formula (I) are intimately admixed with a pharmaceutical carrier according to conventional pharmaceutical compounding techniques, which carrier may take a wide variety of forms depending on the form of preparation desired for administration, e.g., i.v. sterile injectable formulations will be prepared using appropriate solubilizing agents. A unit dose would contain about 15 to 200 mg of the active ingredient. Topiramate is currently available for oral administration in round tablets containing 25 mg, 100 mg or 200 mg of active agent. The tablets contain some or all of the following inactive ingredients: lactose hydrous, pregelatinized starch, microcrystalline cellulose, sodium starch glycolate, magnesium stearate, purified water, carnauba wax, hydroxypropyl methylcellulose, titanium dioxide, polyethylene glycol, synthetic iron oxide, and polysorbate 80.

Wherein the present invention is directed to pharmaceutical administration of one or more compounds of formula (I), the compound(s) of formula (I) may be administered by any suitable method, as would be apparent to one skilled in the art. More particularly, the compound(s) of formula (I) may be administered by any parenteral method including, but not limited to oral, pulmonary, intraperitoneal (ip), intravenous (iv), intramuscular (im), subcutaneous (sc), transdermal, buccal, nasal, sublingual, and rectal. It will be readily apparent to those skilled in the art that any dose or frequency of administration that provides the therapeutic effect described herein is suitable for use in the present invention.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

What is claimed is:

1. A method for treating restless limb syndrome in a subject in need thereof comprising administration of a therapeutically effective amount of a compound of the formula I: 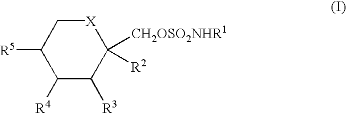
wherein
X is CH₂ or oxygen;
R¹ is hydrogen or alkyl; and
R², R³, R⁴ and R⁵ are independently hydrogen or lower alkyl and, when X is CH₂, R⁴ and R⁵ may be alkene groups joined to form a benzene ring and, when X is oxygen, R² and R³ and/or R⁴ and R⁵ together may be a methylenedioxy group of the following formula (II): 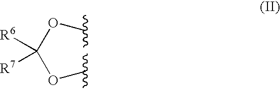
wherein
R⁶ and R⁷ are the same or different and are hydrogen, lower alkyl or are alkyl and are joined to form a cyclopentyl or cyclohexyl ring.

2. The method of claim 1 wherein the compound of formula I is topiramate.

3. The method of claim 2, wherein the therapeutically effective amount topiramate is from about 10 to about 650 mg daily.

4. The method of claim 3, wherein the therapeutically effective amount of topiramate is from about 25 to about 325 mg once or twice daily.

5. The method of claim 1, wherein the restless limb syndrome is restless legs syndrome.

6. The method of claim 1, wherein the restless limb syndrome is drug-induced or drug-exacerbated restless limb syndrome.

7. A method for treating periodic limb movement disorder in a subject in need thereof comprising administration of a therapeutically effective amount of a compound of the formula I: 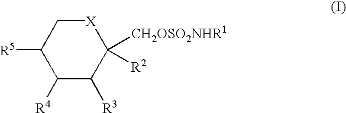
wherein
X is CH₂ or oxygen;
R¹ is hydrogen or alkyl; and
R², R³, R⁴ and R⁵ are independently hydrogen or lower alkyl and, when X is CH₂, R⁴ and R⁵ may be alkene groups joined to form a benzene ring and, when X is oxygen, R² and R³ and/or R⁴ and R⁵ together may be a methylenedioxy group of the following formula (II): 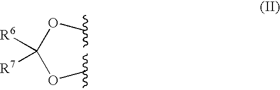
wherein
R⁶ and R⁷ are the same or different and are hydrogen, lower alkyl or are alkyl and are joined to form a cyclopentyl or cyclohexyl ring.

8. The method of claim 7 wherein the compound of formula I is topiramate.

9. The method of claim 8, wherein the therapeutically effective amount topiramate is from about 10 to about 650 mg daily.

10. The method of claim 9, wherein the therapeutically effective amount of topiramate is from about 25 to about 325 mg once or twice daily.

11. The method of claim 7, wherein the periodic limb movement disorder is drug induced or drug exacerbated periodic limb movement disorder.

12. A method for treating sleep disturbances associated with restless limb syndrome or periodic limb movement disorder in a subject in need thereof comprising administration of a therapeutically effective amount of a compound of the formula I: 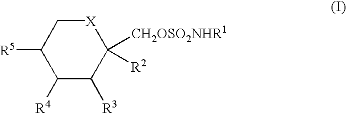
wherein
X is CH₂ or oxygen;
R¹ is hydrogen or alkyl; and
R², R³, R⁴ and R⁵ are independently hydrogen or lower alkyl and, when X is CH₂, R⁴ and R⁵ may be alkene groups joined to form a benzene ring and, when X is oxygen, R² and R³ and/or R⁴ and R⁵ together may be a methylenedioxy group of the following formula (II): 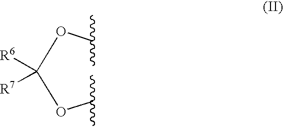
wherein
R⁶ and R⁷ are the same or different and are hydrogen, lower alkyl or are alkyl and are joined to form a cyclopentyl or cyclohexyl ring.

13. The method of claim 12 wherein the compound of formula I is topiramate.

14. The method of claim 13, wherein the therapeutically effective amount topiramate is from about 10 to about 650 mg daily.

15. The method of claim 14, wherein the therapeutically effective amount of topiramate is from about 25 to about 325 mg once or twice daily.
